(19) United States
(12) Patent Application Publication
(10) Pub. No.: US 2026/0153501 A1
(43) Pub. Date: Jun. 4, 2026

(54) METHOD OF PRODUCING PARTICLE FOR IMMUNOTURBIDIMETRY, PARTICLE FOR IMMUNOTURBIDIMETRY, REAGENT, TEST KIT, AND DETECTION METHOD

(71) Applicant: CANON KABUSHIKI KAISHA, Tokyo (JP)

(72) Inventors: GOTA KASAI, Tokyo (JP); RYO NATORI, Tokyo (JP); KENGO KANAZAKI, Kanagawa (JP); FUMIO YAMAUCHI, Kanagawa (JP)

(21) Appl. No.: 19/396,501

(22) Filed: Nov. 21, 2025

(30) Foreign Application Priority Data
Nov. 29, 2024 (JP) 2024-208145

(51) Int. Cl. G01N 33/531

(52) U.S. Cl. CPC G01N 33/531

(57) ABSTRACT

Provided are a method of producing a particle for immunoturbidimetry having a high refractive index and a satisfactory particle size distribution, a particle for immunoturbidimetry, a reagent, a test kit, and a detection method. Specifically, provided are a method of producing a particle for immunoturbidimetry, the method including: a core particle-forming step of subjecting a monomer to emulsion polymerization to form a core particle; a metal oxide layer-forming step of dispersing the core particle in a liquid containing an alcohol-based medium to provide a particle having a metal oxide layer formed on a surface of the core particle by a sol-gel method using a metal alkoxide; and a crystallization step of dispersing the particle having the metal oxide layer formed thereon in an aqueous medium to crystallize the metal oxide layer, and are a reagent, a test kit, and a detection method each using the particle.

### BACKGROUND

### Field of the Technology

The present disclosure relates to a method of producing a particle for immunoturbidimetry, a particle for immunoturbidimetry, a reagent, a test kit, and a detection method.

### Description of the Related Art

In recent years, immunoturbidimetry has been drawing attention as a simple and rapid immunological testing method. There is known a method including mixing a dispersion of a particle having an antibody or an antigen as a ligand on its surface and a specimen that may contain a target substance (antigen or antibody). At this time, when the specimen contains the target substance (antibody or antigen), an aggregation reaction of the particle occurs, and hence the presence or absence of a disease can be identified by optically detecting the aggregation reaction as a variation in, for example, scattered light intensity, transmitted light intensity, or absorbance.

A polystyrene-based latex particle containing polystyrene as a main component has hitherto been used as a particle for immunoturbidimetry because the polystyrene-based latex particle is easy for sensitization (immobilization) of an antigen or an antibody and is relatively inexpensive, and its polymerization reaction is easy to control.

For example, there has been developed a particle using titanium oxide, which is well known as a high-refractive-index material. Japanese Patent Laid-Open No. 2008-241357 proposes a particle for immunoturbidimetry in which a ligand is bound to a particle obtained by coating a carboxylic acid-modified polystyrene particle with a titanium oxide fine particle.

### SUMMARY

The immunoturbidimetry using the polystyrene-based latex particle has failed to detect a trace component in a low-concentration region in some cases. Accordingly, the development of a particle having sensitivity higher than that of the polystyrene-based latex particle has been demanded.

In order to solve the above-mentioned problem, it is required to increase a change in absorbance caused by particle aggregation along with the formation of an immune complex. One factor for controlling the change in absorbance is known to be the refractive index of a component for forming a particle, and hence there has been developed a particle using a high-refractive-index material.

However, it has been found that, in the method including attaching the titanium oxide fine particle described in Japanese Patent Laid-Open No. 2008-241357, the sensitivity when used as a reagent for immunoturbidimetry is insufficient, and a large coefficient of variation resulting from variation in characteristics between particles, such as a particle size distribution and the electronegativity of a surface of a particle, occurs in measurement. The present disclosure is directed to providing a method of producing a particle for immunoturbidimetry that can detect a trace component in a low-concentration region and reduce measurement variation in immunoturbidimetry.

The inventors have found a method capable of producing a particle for immunoturbidimetry that can detect a trace component in a low-concentration region and reduce measurement variation by forming a metal oxide layer outside of a core of a particle, that is, a core particle, using a metal alkoxide, and crystallizing the metal oxide layer, and have arrived at the present disclosure.

That is, according to a first aspect of the present disclosure, there is provided a method of producing a particle for immunoturbidimetry, the method including: a core particle-forming step of subjecting a monomer to emulsion polymerization to form a core particle; a metal oxide layer-forming step of dispersing the core particle in a liquid containing an alcohol-based medium to provide a particle having a metal oxide layer formed on a surface of the core particle by a sol-gel method using a metal alkoxide; and a crystallization step of dispersing the particle having the metal oxide layer formed thereon in an aqueous medium to crystallize the metal oxide layer.

In addition, according to a second aspect of the present disclosure, there is provided a particle for immunoturbidimetry, which is produced by the above-mentioned production method.

In addition, according to a third aspect of the present disclosure, there is provided a reagent including the particle for immunoturbidimetry and an aqueous solution, wherein the particle for immunoturbidimetry is dispersed in the aqueous solution.

Further, according to a fourth aspect of the present disclosure, there is provided a test kit including the reagent and a container configured to accommodate the reagent.

In addition, according to a fifth aspect of the present disclosure, there is provided a method of detecting a target substance in a specimen by in vitro diagnosis, the method including mixing the reagent and a specimen that may contain the target substance.

Finally, according to a sixth aspect of the present disclosure, there is provided a method of detecting a target substance in a specimen by in vitro diagnosis, the method including: mixing the reagent and a specimen that may contain the target substance to provide a mixed solution; irradiating the mixed solution with light; and detecting at least one of transmitted light or scattered light from the light with which the mixed solution has been irradiated.

Features of the present disclosure will become apparent from the following description of embodiments with reference to the attached drawings. The following description of embodiments is described by way of example.

### BRIEF DESCRIPTION OF THE DRAWINGS

FIG. 1 shows a measurement result of an X-ray diffraction pattern of a metal oxide layer-forming particle 1.

FIG. 2 shows a measurement result of an X-ray diffraction pattern of a particle 1.

### DESCRIPTION OF THE EMBODIMENTS

Embodiments of the present disclosure are described in detail below. However, the technical scope of the present disclosure is not limited to the embodiments.

A method of producing a particle for immunoturbidimetry of the present disclosure is characterized by including:
(1) a core particle-forming step of subjecting a monomer to emulsion polymerization to form a core particle;
(2) a metal oxide layer-forming step of dispersing the core particle in a liquid containing an alcohol-based medium to provide a particle having a metal oxide layer formed on a surface of the core particle by a sol-gel method using a metal alkoxide; and
(3) a crystallization step of dispersing the particle having the metal oxide layer formed thereon in an aqueous medium to crystallize the metal oxide layer.

In immunoturbidimetry, it is required to increase a change in absorbance caused by particle aggregation along with the formation of an immune complex in order to detect a trace component in a low-concentration region. The inventors have conceived that the formation of a titanium oxide layer on a core particle and an increase in refractive index of the titanium oxide layer by crystallization enable the detection of a trace component in a low-concentration region and a reduction in measurement variation, and have arrived at the present disclosure.

The core particle of the present disclosure may be obtained by any one of general polymer particle production methods, such as an emulsion polymerization method, a soap-free polymerization method, a dispersion polymerization method, a suspension polymerization method, a phase inversion emulsification method, a wet pulverization method, and a dry pulverization method. Although the production method is not particularly limited, a particle obtained by the emulsion polymerization method and the soap-free polymerization method is preferred because a desired volume average particle diameter for a particle for immunoturbidimetry is obtained and a uniform particle size distribution for a particle for immunoturbidimetry is obtained.

The monomer is not particularly limited, but is, for example, preferably a monomer having a high refractive index. Specific preferred structures for this purpose may include a styrene structure, a fluorene structure, a dinaphthothiophene structure, a naphthalene structure, an anthracene structure, and a phenanthrene structure.

Specifically, the monomer has a repeating unit represented by the formula (1):

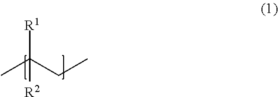

where R₁ represents a hydrogen atom or a methyl group, and R₂ represents a structure having a phenyl group that may be substituted (e.g., styrene) or a group containing an ester bond.

A form of the particle for immunoturbidimetry produced by the production method of the present disclosure is characterized in that the particle for immunoturbidimetry includes an organic polymer and a metal oxide, and includes a first layer (core layer) containing a first organic polymer and a second layer (metal oxide layer) containing the metal oxide, and the second layer is arranged outside of the first layer.

The first layer containing the first organic polymer in the particle for immunoturbidimetry of the present disclosure preferably further has a crosslinked structure. The crosslinked structure is obtained by polymerization using a crosslinkable radical polymerizable monomer, which is a monomer having two or more radical polymerizable unsaturated bonds in one molecule. Examples of such crosslinkable monomer may include polyfunctional (meth)acrylates, such as ethylene glycol diacrylate, ethylene glycol dimethacrylate, trimethylolpropane triacrylate, trimethylolpropane trimethacrylate, pentaerythritol triacrylate, pentaerythritol trimethacrylate, dipentaerythritol hexaacrylate, and dipentaerythritol hexamethacrylate, conjugated diolefins, such as butadiene and isoprene, divinylbenzene, diallyl phthalate, allyl acrylate, and allyl methacrylate. In addition, two or more kinds of crosslinkable radical polymerizable monomers may be used. As the crosslinked structure, a structure represented by the formula (2) is more preferred. When the first layer has the crosslinked structure, the particle becomes physically strong, and there is no concern about cracking or chipping even when centrifugation is repeated during purification:

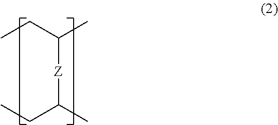

where Z represents a substituted or unsubstituted phenylene group or naphthalene group, provided that, in the case of substitution, a substituent is a methyl group or an ethyl group, and Z may be different among structural units.

Examples of the crosslinkable radical polymerizable monomer to be used to form the crosslinked structure of the formula (2) include 1,2-divinylbenzene, 1,3-divinylbenzene, 1,4-divinylbenzene, 2,6-diethynylnaphthalene, and 2,7-diethynylnaphthalene. Those monomers may be used alone or a plurality thereof may be used at the same time.

Of the crosslinkable radical polymerizable monomers given as examples, divinylbenzene is preferred. Although the reason is not clear, when divinylbenzene is used, handleability during a radical polymerization reaction is excellent, and a monomer conversion rate during particle formation is improved.

The metal oxide layer of the present disclosure may be obtained by a sol-gel method in which a metal oxide precursor is hydrolyzed. That is, the metal oxide layer may be obtained by allowing a metal oxide precursor, an alcohol-based medium, and water to coexist and performing a hydrolysis reaction.

Examples of the metal oxide precursor include a metal chloride, a metal acetate, a metal alkoxide, and a metal hydroxide. Of those, a metal alkoxide, a metal acetate, and a metal hydroxide are preferably used from the viewpoint of impurities (e.g., a chloride) that are by-produced. Of those, a metal alkoxide represented by the chemical formula Mₓ(OR)_{y} (where M represents a metal element, R represents an alkyl group, and “x” and “y” each independently represent an integer of 1 or more and 4 or less), a metal hydroxide represented by the chemical formula Mₓ(OH)_{y}·H₂O (where M represents a metal element, “x” and “y” each independently represent an integer of 1 or more and 4 or less, and “n” represents an integer of 1 or more), and a compound containing the above-mentioned metal alkoxide and/or metal hydroxide are particularly preferred.

In the present disclosure, a titanium alkoxide is more preferred. Specific examples thereof include titanium methoxide, titanium ethoxide, titanium diisopropoxide bis(2,4-pentanedionate), titanium diisopropoxide bis(ethyl acetoacetate), titanium n-butoxide, titanium isopropoxide, titanium methoxypropoxide, titanium n-nonyloxide, titanium n-propoxide, titanium stearyloxide, titanium triisostearyl isopropoxide, and titanium trimethylsiloxide.

Examples of the alcohol-based medium include ethanol, 1-propanol, 2-propanol, and butanol.

Further, in the metal oxide layer-forming step, it is preferred that the alcohol-based medium contain a nonionic water-soluble polymer. The addition of the nonionic water-soluble polymer to form the metal oxide layer enables the suppression of particle aggregation during the formation of the metal oxide layer, and hence the measurement variation can be further reduced. Examples of the nonionic water-soluble polymer include polyvinylpyrrolidone, polyethyleneimine ethoxylate, and polyvinyl alcohol.

In the present disclosure, the term “crystallization” means the formation of an arrangement called a crystal lattice, which is a repeated arrangement of unit cells of a metal crystal with respect to a metal oxide. When X-ray diffraction is performed on the metal crystal, a crystal structure can be determined by the presence or absence of a peak, and a ratio of a crystallized metal oxide (also referred to as “degree of crystallinity” in the present disclosure) can also be calculated from the size of a peak area. In the crystallization step of the metal oxide layer of the present disclosure, it is preferred that the temperature of the aqueous medium be increased to 50° C. or more. When the temperature of the aqueous medium is set within the range, titanium oxide can be partially crystallized from an amorphous state. Through this treatment, the refractive index of the particle is improved, and detection sensitivity in immunoturbidimetry can be improved.

The pH of the aqueous medium in the crystallization step of the metal oxide layer of the present disclosure is 6 or more and 9 or less, and it is preferred that the aqueous medium further contain a salt in the concentration range of 1 mM or more and 100 mM or less. The suppression of the pH fluctuation along with a temperature change of a metal oxide layer-forming particle dispersion can suppress the aggregation of a metal oxide layer-forming particle in the crystallization step, and hence the measurement variation can be further reduced.

The particle for immunoturbidimetry of the present disclosure may include a third layer (organic layer) containing a second organic polymer formed outside of the metal oxide layer. When the organic layer is formed, the adsorption rate of an antibody or an antigen serving as a biosensor is improved, and higher sensitivity can be achieved.

The structure of a resin of the organic layer of the present disclosure is not particularly limited as long as the resin has a repeating unit represented by the formula (3):

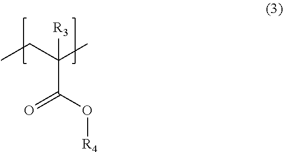

where R₃ represents a hydrogen atom or a methyl group, R₄ represents a group having an epoxy group, a group having a hydroxy group, or a group having a carboxy group, and R₃ and R₄ may be different among structural units.

The formula (3) preferably represents a structure represented by the formula (3-A). The structure represented by the formula (3-A) has any one of a hydroxy group or a carboxy group. Accordingly, an ability to suppress non-specific adsorption is equal to or higher than that of a structure having an epoxy group, and hence the structure is preferred. In addition, it is conceived that, when the structure of a resin of the organic layer has any one of a hydroxy group or a carboxy group, a hydrogen bond can be formed with titanium oxide to allow for satisfactory interaction:

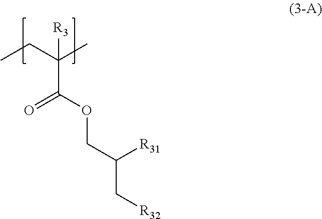

where any one of R₃₁ or R₃₂ represents a hydroxy group, and the other represents a hydroxy group or a group represented by the formula (3-B):

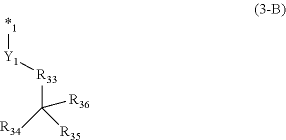

where R₃₃ represents a single bond or a methylene group, R₃₄, R₃₅, and R₃₆ each represent a hydrogen atom, a methyl group, a hydroxy group, a carboxy group, a hydroxymethyl group, or a carboxymethyl group, and at least one of R₃₄, R₃₅, or R₃₆ includes a hydroxy group or a carboxy group, Y₁ represents a sulfur atom or an imino group, and *1 represents a bonding position with a structure represented by the formula (3-A).

It is conceived that the organic layer contains such a vinyl-based polymer as represented by the formula (3), and hence uniform coating can be performed without exposure of titanium oxide. That is, the coating resin satisfactorily interacts with titanium oxide and uniform coating can be performed without exposure of titanium oxide. Accordingly, the desorption of the resin can be suppressed and a decrease in water dispersion stability of the particle for immunoturbidimetry can be suppressed. Specific examples of the structure of the formula (3) are shown in the following formulae (3-A-1) to (3-A-12), but the present disclosure is not limited thereto.

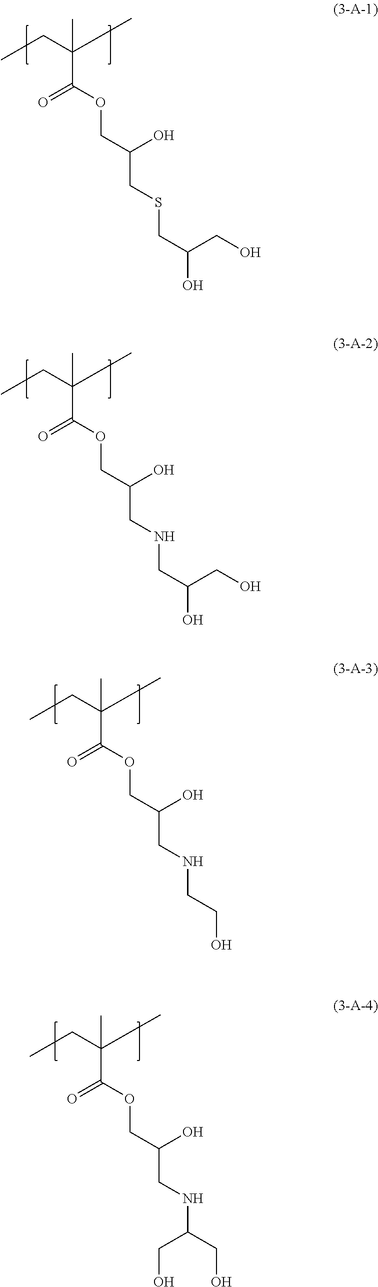

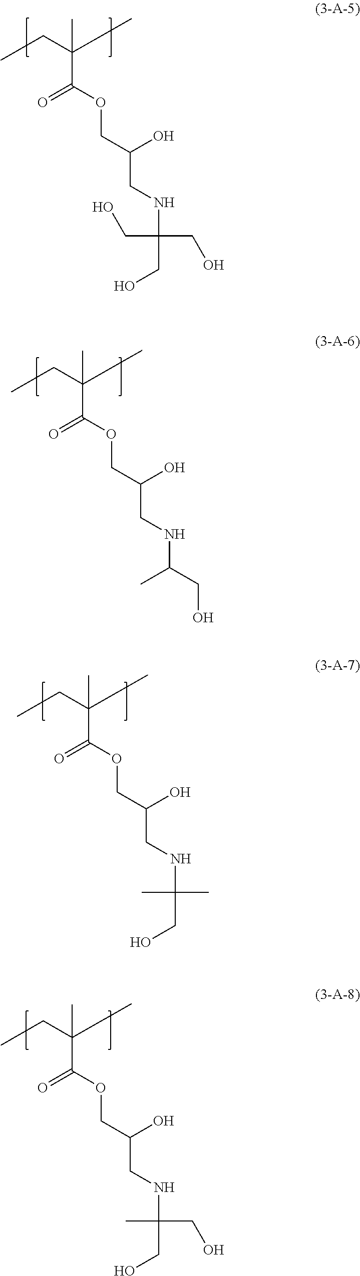

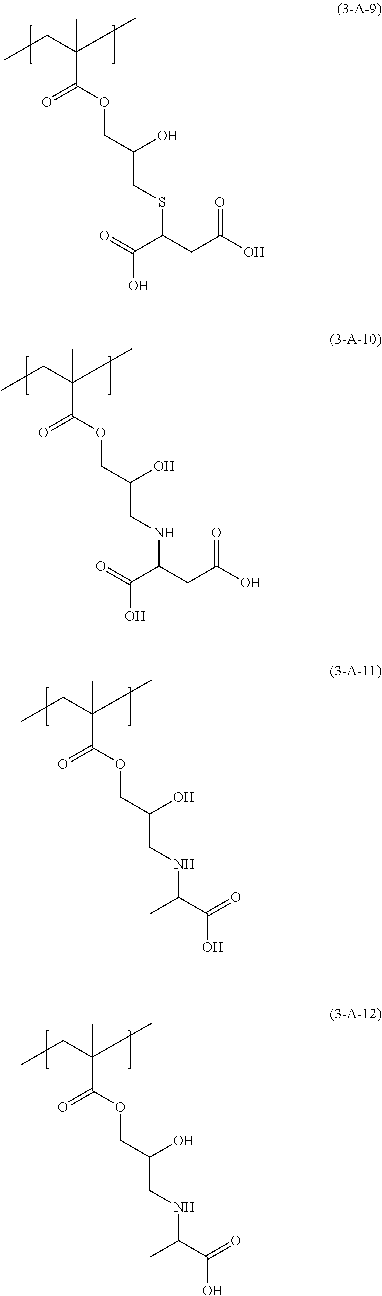

Further, the organic layer preferably further has a repeating unit represented by the formula (4) or the formula (5) because the organic layer contains a vinyl-based polymer and an alkoxysilane, and hence the exposure of titanium oxide is further suppressed by interaction with titanium oxide, and uniform coating is achieved. A specific example thereof is a repeating unit derived from vinyltrimethoxysilane, vinyltriethoxysilane, 3-methacryloxypropylmethyldimethoxysilane, 3-methacryloxypropyltrimethoxysilane, 3-methacryloxypropylmethyldiethoxysilane, 3-methacryloxypropyltriethoxysilane, or 3-acryloxypropyltrimethoxysilane.

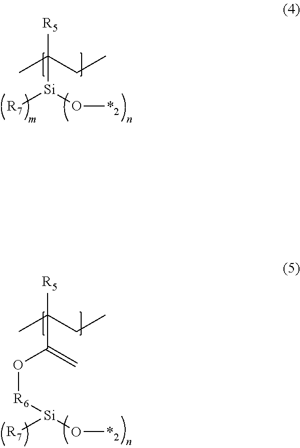

R₅ represents a hydrogen atom or a methyl group, R₆ represents a single bond, a phenylene group, or an alkylene group having 3 or less carbon atoms, “n” represents an integer of 1 or more and 3 or less, “m” represents an integer of 0 or more and 2 or less, n+m is 3, *_{2S} each independently represent a bond to a titanium atom or a silicon atom, or represent a hydrogen atom, a methyl group, or an ethyl group, and R_{7S} each independently represent a methyl group or an ethyl group. That is, the structure represented by each of the formula (4) and the formula (5) may be bonded to a titanium atom of titanium oxide via an oxygen atom, or may be bonded to a silicon atom of another structure represented by the formula (4) or the formula (5) via an oxygen atom.

The organic layer of the present disclosure may also be obtained by any one of general polymer particle production methods, such as an emulsion polymerization method, a soap-free polymerization method, a dispersion polymerization method, a suspension polymerization method, a phase inversion emulsification method, a wet pulverization method, and a dry pulverization method in the same manner as that for the core particle. Although the production method is not particularly limited, a particle obtained by the emulsion polymerization method or the soap-free polymerization method is preferred because a uniform particle size distribution for a particle for immunoturbidimetry is obtained.

(Method of measuring Content of Titanium Oxide in Particle)

A method of measuring the content of titanium oxide in the particle in the present disclosure is described. The content of titanium oxide in the particle is measured with a thermogravimetric measuring device. For example, NEXTA (trademark) STA200 (Hitachi High-Tech Science Corporation) is used. 5 mg to 10 mg of a cured product was weighed in an aluminum pan and measured in a nitrogen atmosphere. The temperature conditions were to hold the temperature at 30° C. for 30 minutes, then increase the temperature from 30° C. to 500° C. at 10° C./min, and subsequently hold the temperature at 500° C. for 10 minutes.

As an evaluation, the content of titanium oxide in the particle was measured from the mass loss of a resin component at 300° C.

(Method of measuring Crystallization of Particle)

The crystallization of the particle is measured with X′Pert-Pro (Malvern Panalytical Ltd.). An X-ray diffraction pattern is measured in the range of 20°<20≤60° under the conditions of an X-ray output of 45 kV and 40 mA. The ratio of a peak area showing a crystal component to a total measured peak area is calculated as a degree of crystallinity and evaluated.

(Methods of measuring Volume Average Particle Diameter and Particle Size Distribution of Particles in Aqueous Dispersion)

A method of measuring the volume average particle diameter (Dv) of particles in the present disclosure is described. The Dv of particles present in an aqueous dispersion is measured by a dynamic light scattering method. For example, Zetasizer (Zetasizer ultra: manufactured by Malvern Panalytical Ltd.) is used and measurement is performed at 25° C.

In addition, the particle size distribution of the particles in the present disclosure is calculated by measuring a number average particle diameter (Dn) by the above-mentioned dynamic light scattering method to determine the ratio of Dv to Dn (Dv/Dn).

### (Particle for Immunoturbidimetry)

The particle for immunoturbidimetry can be produced by the production method of the present disclosure. The particle for immunoturbidimetry may be an affinity particle for detecting a target substance, including a ligand on its surface.

(Substance that Specifically Binds to Target Substance (Ligand))

The ligand is a compound that specifically binds to a receptor of a specific target substance. A site where the ligand binds to the target substance is fixed, and has selectively or specifically high affinity. Examples thereof include an antigen and an antibody, an enzyme protein and a substrate therefor, a signal substance, such as a hormone or a neurotransmitter, and a receptor therefor, a nucleic acid, and avidin and biotin, but the present disclosure is not limited thereto. Specific examples of the ligand include an antigen, an antibody, an antigen-binding fragment (e.g., Fab, F(ab′)₂, F(ab′), Fv, or scFv), a naturally derived nucleic acid, an artificial nucleic acid, an aptamer, a peptide aptamer, an oligopeptide, an enzyme, and a coenzyme.

The ligand in the particle for immunoturbidimetry of the present disclosure is preferably an antibody or a virus-derived antigen. When the ligand is an antibody or a virus-derived antigen, a target substance that binds to the antibody or the antigen can be detected with high sensitivity. In the present disclosure, a method of immobilizing the ligand on the particle may be performed by any known method, and the ligand can be immobilized by physically or chemically bonding the ligand to the particle. Examples of a method for the chemically bonding include a carbodiimide-mediated reaction, an NHS ester activation reaction, and a method of bonding a biotin-modified ligand to an avidin-bonded carboxy group.

### (Reagent)

The particle for immunoturbidimetry of this embodiment may be a reagent for detecting a target substance via a ligand. Although a form as a reagent is not limited, it is preferred to use a reagent in which the particle for immunoturbidimetry of the present disclosure is dispersed in an aqueous solution.

### (Test Kit)

The reagent including the particle for immunoturbidimetry of this embodiment may be a test kit for detecting a target substance via a ligand. Although a form as a test kit is not limited, the test kit preferably includes a reagent including the particle for immunoturbidimetry of the present disclosure and a container configured to accommodate the reagent.

The composition of the reagent is not particularly limited, but as an example, a form including a buffer solution and a surfactant as a first reagent, and including a buffer solution, a surfactant, and a particle for immunoturbidimetry as a second reagent is preferred.

### (Detection Method)

The reagent including the particle for immunoturbidimetry of this embodiment can detect a target substance in a specimen by in vitro diagnosis. The term “detection” in the present disclosure may have both the meanings of qualitative analysis and quantitative analysis of the target substance. A method of detecting a target substance using the reagent including the particle for immunoturbidimetry of this embodiment is not particularly limited, but as an example, a method including the following three steps is preferred:
(1) mixing the reagent including the particle for immunoturbidimetry of this embodiment with a specimen that may contain the target substance to provide a mixed solution;
(2) irradiating the mixed solution with light; and
(3) detecting at least one of transmitted light or scattered light from the light with which the mixed solution has been irradiated.

### EXAMPLES

The present disclosure is described in detail below by way of Examples, but the present disclosure is not limited to these Examples.

### Particle Preparation Example 1

### (Step-1/Core Particle-Forming Step)

12.68 g of styrene (St: Kishida Chemical Co., Ltd.), 0.23 g of divinylbenzene (DVB: Kishida Chemical Co., Ltd.), and 1,512.02 g of ion-exchanged water were weighed into a 2 L four-necked separable flask to prepare a mixed solution. Oxygen was removed from the inside of the four-necked separable flask by holding the mixed solution at 70° C. under stirring at 140 rpm and performing a nitrogen flow at a flow rate of 200 ml/min. Next, a dissolved solution of 0.55 g of V-50 (FUJIFILM Wako Pure Chemical Corporation) dissolved in 20 g of ion-exchanged water, which had been separately prepared, was added to the mixed solution to start soap-free emulsion polymerization. A dispersion of a core particle 1 containing a copolymer of St and DVB was obtained by a reaction for 23 hours from the start of the polymerization. Part of the dispersion was collected and evaluated by using the dynamic light scattering (Zetasizer ultra: Malvern Panalytical Ltd.) of the core particle 1, and as a result, the volume average particle diameter was 190 nm.

### (Step-2/Metal Oxide Layer-Forming Step)

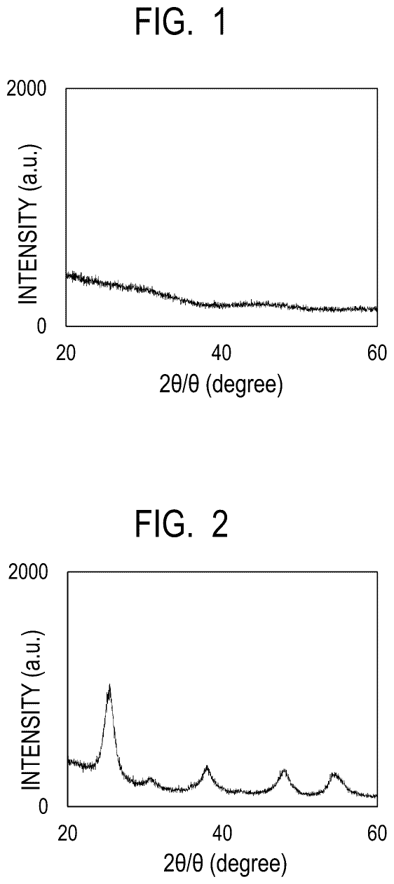

A dispersion was prepared with ion-exchanged water so that 1 g of a core particle 1 dispersion having a solid content concentration of 0.6% by mass was obtained. The dispersion was mixed into 20.94 g of ethanol (Kishida Chemical Co., Ltd.) containing 0.2% by mass of polyvinylpyrrolidone K-30 (PVP K-30: Kishida Chemical Co., Ltd.), and the mixture was held at 70° C. while being stirred at 800 rpm. Next, a solution of 125 μL of titanium (IV) n-butoxide, monomer (TBOT: Kishida Chemical Co., Ltd.) mixed into 9.88 g of ethanol, which had been separately prepared, was added to the mixed solution to start a sol-gel reaction. The reaction was performed for 6 hours from the start of the sol-gel reaction, and a particle was separated from the mixed solution as a metal oxide layer-forming particle 1 with a centrifuge, and was re-dispersed in ethanol. Further, the operation of separating the metal oxide layer-forming particle 1 from the dispersion with a centrifuge and re-dispersing the particle 1 in ion-exchanged water was repeated twice to purify the metal oxide layer-forming particle 1, and finally, the metal oxide layer-forming particle 1 was stored in a state of an aqueous dispersion prepared so that the concentration was 5.0% by mass. Part of the dispersion was collected and the dynamic light scattering of the metal oxide layer-forming particle 1 was evaluated, and as a result, the volume average particle diameter was 200 nm. In addition, when the metal oxide content was evaluated by using a differential thermal-thermogravimetric simultaneous analysis (NEXTA (trademark) STA200RV: Hitachi High-Tech Corporation), the content was 43% by mass of the particle mass. Further, when the crystallinity was evaluated by X-ray diffraction, no crystallization peak was observed in the metal oxide layer-forming particle 1 as shown in the result of FIG. 1.

### (Step-3/Metal Oxide Layer-Crystallizing Step)

A dispersion was prepared with an N-(2-hydroxyethyl) piperazine-N′-2-ethanesulfonic acid (HEPES: Kishida Chemical Co., Ltd.) buffer solution, which had been prepared in advance, so that 10 g of a metal oxide layer-forming particle 1 dispersion having a solid content concentration of 0.5% by mass was obtained. The HEPES buffer solution used was a 10 mM HEPES buffer solution adjusted to a pH of 7.86 with a 1 N aqueous sodium hydroxide solution. The metal oxide layer-forming particle 1 dispersion was held at 70° C. while being stirred at 800 rpm to start the crystallization of the metal oxide layer. The reaction was performed for 24 hours from the start of the crystallization to provide a particle 1 in which the metal oxide layer was crystallized. Part of the dispersion was collected and the dynamic light scattering of the particle 1 was evaluated, and as a result, the volume average particle diameter was 200 nm.

Further, when the crystallinity was evaluated by X-ray diffraction, a peak was observed as shown in the result of FIG. 2. This indicates that part of the titanium oxide layer on the surface of the particle 1 was crystallized and had an anatase-type crystal structure.

### Particle Preparation Example 2

### (Step-1/Core Particle-Forming Step)

A core particle 2 was prepared by Step 1 described in Example 1.

### (Step-2/Metal Oxide Layer-Forming Step)

A metal oxide layer-forming particle 2 was prepared by Step 2 described in Example 1.

### (Step-3/Organic Layer-Forming Step and Metal Oxide Layer-Crystallizing Step)

A dispersion was prepared with ion-exchanged water so that 149.55 g of a metal oxide layer-forming particle 2 dispersion having a solid content concentration of 0.2% by mass was obtained. 0.135 g of glycidyl methacrylate (GMA: Kishida Chemical Co., Ltd.) and 0.015 g of 3-methacryloxypropyltrimethoxysilane (MPS: Shin-Etsu Chemical Co., Ltd.) were added thereto, and the mixture was held at 70° C. while being stirred at 100 rpm. Oxygen was removed from the inside of the four-necked separable flask by performing a nitrogen flow at a flow rate of 200 mL/min. Then, a dissolved solution of 0.03 g of V-50 in 0.3 g of ion-exchanged water, which had been separately prepared, was added to the mixed solution to start shell formation. A dispersion containing an organic layer-forming particle 2 having a core, a metal oxide layer, and a shell structure was obtained by continuing stirring for 18 hours after the start of the reaction. Part of the dispersion was taken out and the crystallinity was evaluated by X-ray diffraction, and as a result, a peak showing an anatase-type crystal structure was observed.

### (Step-4/Functional Group-Imparting Step)

An aqueous solution in mercaptosuccinic acid (MSA: FUJIFILM Wako Pure Chemical Corporation) was dissolved, which had been prepared in advance, was added to the dispersion containing the organic layer-forming particle 2. At this time, an aqueous solution prepared so that the total number of moles of MSA was adjusted to be equal to the number of moles of the glycidyl methacrylate was used. Subsequently, triethylamine (Kishida Chemical Co., Ltd.) was added thereto to adjust the pH to 10. Next, the mixture was heated to 70° C. while being stirred at 800 rpm and further held in this state for 18 hours to provide a dispersion of a particle 2 having a core, a metal oxide layer, a shell structure, and a functional group. The operation of separating the particle 2 from the dispersion with a centrifuge and re-dispersing the particle 2 in ion-exchanged water was repeated eight times to purify the particle 2, and finally, the particle 2 was stored in a state of an aqueous dispersion prepared so that the concentration was 5.0% by mass.

The particle 2 included a styrene-divinylbenzene copolymer, that is, had a structure unit represented by the formula (1) in which R₁ represented a hydrogen atom and R₂ represented a phenyl group in its core. In addition, the particle 2 had a structural unit represented by the formula (3) in which R₃ represented a methyl group and R₄ represented a structural unit represented by any one of the following formula (3-C), formula (3-D), or formula (3-E) in its organic layer:

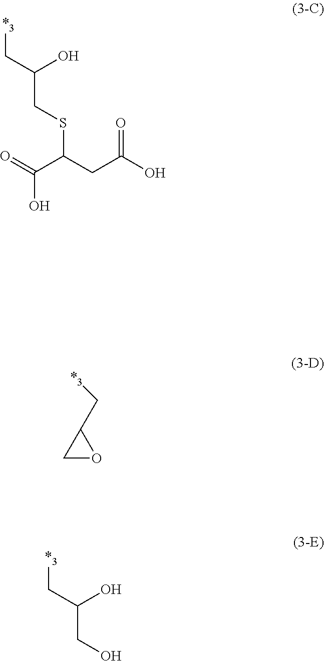

where *₃ represents a bonding position with the structure represented by the formula (3).

### Comparative Particle Preparation Example

### (Step-1/Core Particle Preparation)

100 μL of a carboxy-modified polystyrene fine particle dispersion (IMMUTEX: JSR Corporation), 900 μL of a HEPES buffer solution containing 1% by mass bovine serum, and 500 μL of a soluble carbodiimide solution (1 M aqueous solution of 1-ethyl-3-(3-dimethylaminopropyl) carbodiimide hydrochloride, the same applies hereinafter) were loaded into a centrifuge tube, and the mixture was held at room temperature (20° C. to 30° C.) for 1 hour while being stirred at 100 rpm. Next, the carboxy-modified polystyrene particle was separated from the dispersion with a centrifuge and then re-dispersed in ion-exchanged water. Further, the carboxy-modified polystyrene particle was separated from the dispersion with a centrifuge and re-dispersed in 500 μL of a HEPES buffer solution containing 1% by mass bovine serum to provide a carboxy group-activated polystyrene fine particle dispersion.

### (Step-2/Metal Oxide Layer-forming Step)

50 μL of a titanium oxide fine particle dispersion and 950 μL of a γ-aminopropyltriethoxysilane aqueous solution (ion-exchanged water containing 1% by mass of γ-aminopropyltriethoxysilane) were loaded into a centrifuge tube, and the mixture was held at room temperature (20° C. to 30° C.) for 1 hour while being stirred at 100 rpm. Next, the operation of separating the titanium oxide fine particle from the dispersion with a centrifuge and re-dispersing the titanium oxide fine particle in an acetate buffer solution (0.01 M, pH: 5.0) was repeated three times. The obtained precipitate was re-dispersed in 500 μL of a phosphate buffer solution (0.01 M, pH: 6.0) to provide an amino group-introduced titanium oxide fine particle dispersion.

88 μL of the amino group-introduced titanium oxide fine particle dispersion and 500 μL of the carboxy group-activated polystyrene fine particle dispersion were loaded into a centrifuge tube, and the mixture was held at room temperature (20° C. to 30° C.) for 2 hours while being stirred. Next, a titanium oxide-polystyrene composite fine particle was separated from the dispersion with a centrifuge, and the precipitate was re-dispersed in 1,500 μL of a phosphate buffer solution (0.1 M, pH: 7.1) to provide a titanium oxide-polystyrene composite fine particle dispersion.

The titanium oxide-polystyrene composite fine particle dispersion was centrifuged by a density gradient centrifugation method, and a fraction having a density of 1.6 (content of titanium oxide in the composite particle: 20% by mass) was obtained as a comparative particle 1.

### Example 1, Example 2, and Comparative Example

### (Preparation of Particle for Immunoturbidimetry: Particle 1)

In each of Example 1 and Example 2, and Comparative Example, an example of a particle for immunoturbidimetry with ferritin as a target substance is shown.

100 μL (1 mg in terms of particle solid content) of the dispersion of the particle 1 diluted with ion-exchanged water to a solid content concentration of 1.0% by mass was aliquoted into a 1.5 mL microtube. After centrifugal washing, a HEPES buffer solution having a pH of 7.0 was added thereto, and the mixture was dispersed with an ultrasonic wave. 24 μL (0.12 mg in terms of antibody amount) of a 5.0 mg/mL dispersion of a mouse monoclonal anti-ferritin antibody (isoelectric point: 7.1) was added thereto, and the mixture was stirred at room temperature for 1 hour to provide a particle 1 for immunoturbidimetry in which the particle was sensitized with the antibody.

### (Preparation of Particles for Immunoturbidimetry: Particle 2 and Comparative Particle 1)

300 μL (3 mg in terms of particle solid content) of the dispersion of each of the particle 2 and the comparative particle 1 diluted with ion-exchanged water to a solid content concentration of 1.0% by mass was aliquoted into a 1.5 mL microtube. 90 μL of a 5.0% by mass aqueous solution of 1-(3-dimethylaminopropyl)-3-ethylcarbodiimide hydrochloride (Tokyo Chemical Industry Co., Ltd.) and 90 μL of a 5.0% by mass aqueous solution of N-hydroxysulfosuccinimide sodium salt (Tokyo Chemical Industry Co., Ltd.) were added thereto. The mixture was stirred at room temperature for 30 minutes to provide a dispersion of a particle having an activated carboxy group (activated particle dispersion).

After centrifugal washing, 270 μL of phosphate-buffered saline (hereinafter referred to as “PBS”) having a pH of 5.5 was added thereto, and the particle having an activated carboxy group was dispersed with an ultrasonic wave. 24 μL (0.12 mg in terms of antibody amount) of a 5.0 mg/mL dispersion of a mouse monoclonal anti-ferritin antibody (isoelectric point: 7.1) was added thereto, and the mixture was stirred at room temperature for 3 hours to provide a particle 2 for immunoturbidimetry and a comparative particle 1 for immunoturbidimetry in each of which the particle was sensitized with the antibody.

### (Preparation of First Reagent)

A first reagent was obtained by dissolving 50 mM HEPES, 0.05% by mass Triton X-100, and 1.0% by mass sodium chloride (Kishida Chemical Co., Ltd.) in ion-exchanged water.

### (Preparation of Second Reagent)

After the centrifugal washing of the particle 1 for immunoturbidimetry, the resultant was re-dispersed in 500 μL of a buffer (HEPES buffer) in which 10 mM HEPES, 0.01% by mass polyoxyethylene nonylphenyl ether (Triton X-100: Kishida Chemical Co., Ltd.), and 10% by mass sucrose (viscosity modifier) was dissolved in ion-exchanged water. After that, the contents were mixed and diluted with the HEPES buffer so that the concentration of the particle for immunoturbidimetry became 0.1% by mass to provide a second reagent 1.

A second reagent 2 and a second reagent 3 were each prepared by the same experimental operation as in that the preparation of the second reagent 1 except that the kind of the particle was changed from the particle 1 for immunoturbidimetry to the particle 2 for immunoturbidimetry or the comparative particle 1 for immunoturbidimetry.

### (Measurement of Amount of Change in Absorbance)

A mixed solution was prepared by mixing 15 μL of a specimen prepared so that a ferritin concentration was 250 ng/ml and 60 μL of the first reagent 1, and the mixed solution was kept warm for 290 seconds under the condition of 37° C. Next, 30 μL of the second reagent 1 was mixed into the mixed solution, the mixture was stirred, and an absorbance after 42 seconds was measured. Further, the mixed solution was allowed to stand still at 37° C. for 253 seconds, and then an absorbance was measured again, and a difference from the absorbance after 42 seconds was defined as the amount of change in absorbance AAbs. A spectrophotometer BIOSPECTROMETER (Eppendorf) was used for the absorbance measurement, and a measurement wavelength was set to 572 nm.

### (Calculation of Sensitivity Index)

A value of ΔAbs×10,000 was calculated and defined as a ferritin sensitivity index. It is expected that as the ferritin sensitivity index becomes larger, the target substance can be detected with high sensitivity.

An evaluation was performed as described below according to the value of the sensitivity index.
A: The value of ΔAbs×10,000 was larger than 100.
B: The value of ΔAbs×10,000 was 100 or less.

The results for the respective particles are shown in Table 1.

### (Calculation of Sensitivity Variation)

The measurement of the amount of change in absorbance was performed ten times, and a coefficient of variation in all measurements was measured and defined as an index of sensitivity variation. It is expected that as the coefficient of variation becomes smaller, variation between measurements becomes smaller. An evaluation was performed as described below according to the value of the coefficient of variation.
A: The value of the coefficient of variation was smaller than 3%.
B: The value of the coefficient of variation was 3% or more.

The results for the respective particles are shown in Table 1.

TABLE 1

| | Comparative | | |
|---|---|---|---|
| | Example 1 | Example 1 | Example 2 |
| Particle | Comparative | Particle 1 for | Particle 2 for |
| | Particle 1 for | Immuno- | Immuno- |
| | immuno- | turbidimetry | turbidimetry |
| | turbidimetry | | |
| Titanium oxide content | 20% | 40% | 32% |
| | by mass | by mass | by mass |
| Volume average particle | 450 nm | 200 nm | 210 nm |
| diameter | | | |
| Measurement sensitivity | B | A | A |
| Coefficient of variation | B | A | A |

Those results show that the particle for immunoturbidimetry produced by the present disclosure has a large amount of change in absorbance and a small coefficient of variation when the concentration of the target substance is 250 ng/mL. Thus, it was found that the particle produced by the present disclosure was able to detect a trace component in a low-concentration region and reduce measurement variation.

According to the present disclosure, a particle for immunoturbidimetry that can detect a trace component in a low-concentration region and reduce measurement variation can be produced. In addition, a reagent and a kit each using the particle can be provided.

While the present disclosure has been described with reference to embodiments, it is to be understood that the present disclosure is not limited to the disclosed embodiments. The scope of the following claims is to be accorded the broadest interpretation so as to encompass all such modifications and equivalent structures and functions.

This application claims the benefit of Japanese Patent Application No. 2024-208145, filed Nov. 29, 2024, which is hereby incorporated by reference herein in its entirety.

What is claimed is:

1. A method of producing a particle for immunoturbidimetry, the method comprising:
a core particle-forming step of subjecting a monomer to emulsion polymerization to form a core particle;
a metal oxide layer-forming step of dispersing the core particle in a liquid containing an alcohol-based medium to provide a particle having a metal oxide layer formed on a surface of the core particle by a sol-gel method using a metal alkoxide; and
a crystallization step of dispersing the particle having the metal oxide layer formed thereon in an aqueous medium to crystallize the metal oxide layer.

2. The method of producing a particle for immunoturbidimetry according to claim 1, wherein, in the core particle-forming step, the monomer is represented by the following formula (1): 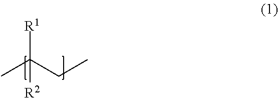
where R₁ represents a hydrogen atom or a methyl group, and R₂ represents a structure having a phenyl group that may be substituted or a group containing an ester bond.

3. The method of producing a particle for immunoturbidimetry according to claim 1, wherein, in the metal oxide layer-forming step, the alcohol-based medium contains a nonionic water-soluble polymer.

4. The method of producing a particle for immunoturbidimetry according to claim 1, wherein, in the metal oxide layer-forming step, a titanium alkoxide is used as the metal alkoxide.

5. The method of producing a particle for immunoturbidimetry according to claim 1, comprising a step of further forming an organic layer on a surface of the particle having the metal oxide layer formed thereon.

6. The method of producing a particle for immunoturbidimetry according to claim 1, wherein the crystallization step includes stirring the aqueous medium in which the particle having the metal oxide layer formed thereon is dispersed and raising a temperature of the aqueous medium to crystallize the metal oxide layer.

7. A particle for immunoturbidimetry, which is produced by the method of claim 1.

8. The particle for immunoturbidimetry according to claim 7, comprising a ligand on a surface thereof.

9. A reagent comprising the particle for immunoturbidimetry of claim 7 and an aqueous solution,
wherein the particle for immunoturbidimetry is dispersed in the aqueous solution.

10. A test kit comprising the reagent of claim 9 and a container configured to accommodate the reagent.

11. A method of detecting a target substance in a specimen by in vitro diagnosis, the method comprising mixing the reagent of claim 9 and a specimen that may contain the target substance.

12. A method of detecting a target substance in a specimen by in vitro diagnosis, the method comprising:
mixing the reagent of claim 9 and a specimen that may contain the target substance to provide a mixed solution;
irradiating the mixed solution with light; and
detecting at least one of transmitted light or scattered light from the light with which the mixed solution has been irradiated.
